(19) United States
(12) Patent Application Publication
(10) Pub. No.: US 2026/0175006 A1
(43) Pub. Date: Jun. 25, 2026

(54) BLOOD PUMPS, RELATED COMPONENTS, METHODS OF USE AND RELATED METHODS OF FABRICATION

(71) Applicant: FBR Medical, Inc., El Dorado Hills, CA (US)

(72) Inventors: Jesus (Jesse) Flores, Mission Viejo, CA (US); Peter G. Piferi, The Villages, FL (US); Andrew J. Boyce, Laguna Woods, CA (US); Larry L. Hood, Irvine, CA (US)

(21) Appl. No.: 18/872,325

(22) PCT Filed: May 8, 2023
(86) PCT No.: PCT/US2023/021351

§ 371 Date: Dec. 6, 2024

Related U.S. Application Data
(60) Provisional application No. 63/352,962, filed on Jun. 16, 2022.
(60) Provisional application No. 63/352,932, filed on Jun. 16, 2022.
(60) Provisional application No. 63/352,907, filed on Jun. 16, 2022.
(60) Provisional application No. 63/353,353, filed on Jun. 17, 2022.
(60) Provisional application No. 63/374,426, filed on Sep. 2, 2022.

(51) Int. Cl. A61M 60/237; A61M 60/13; A61M 60/139; A61M 60/148; A61M 60/178; A61M 60/216; A61M 60/414; A61M 60/419; A61M 60/422; A61M 60/829

(52) U.S. Cl. CPC A61M 60/237; A61M 60/13; A61M 60/139; A61M 60/148; A61M 60/178; A61M 60/216; A61M 60/414; A61M 60/419; A61M 60/422; A61M 60/829

(57) ABSTRACT

Blood pumps configured with a multiple lumen shaft that includes a torque cable with a center lumen that is attached at a first end portion to a motor shaft that is part of an extracorporeal pump motor and is attached at a second axially spaced apart end portion to an intrabody impeller. The multi-lumen shaft includes a torque cable lumen sized and configured to encase the torque cable, at least one in-flow lumen and at least one out-flow lumen.

### RELATED APPLICATIONS

This patent application claims priority to and the benefit of priority to U.S. Provisional Application Ser. No. 63/374,426, filed Sep. 2, 2022, U.S. Provisional Application Ser. No. 63/352,907, filed Jun. 16, 2022, U.S. Provisional Application Ser. No. 63/352,932, filed Jun. 16, 2022, U.S. Provisional Application Ser. No. 63/352,962, filed Jun. 16, 2022, and, U.S. Provisional Application Ser. No. 63/353,353, filed Jun. 17, 2022, the contents of which are hereby incorporated by reference as if recited in full herein.

### FIELD OF THE INVENTION

The present invention relates to blood pumps and is particularly suitable for intra-vascular blood pumps such as cardiac blood pumps.

### BACKGROUND

Over the years, various types of blood pumps have been developed for the purpose of augmenting or replacing the blood pumping action of damaged or diseased hearts. The pumps may be designed to provide right and/or left ventricular assist, although left ventricle assist is the most common application in that it is far more common for the left ventricle to become diseased or damaged than it is for the right ventricle.

Blood pumps must pump the fluid at a suitable rate without applying excessive Reynolds shear stress to the fluid. It is well known to those skilled in the art that lysis or cell destruction may result from application of shear stress to cell membranes. Red blood cells are particularly susceptible to shear stress damage as their cell membranes do not include a reinforcing cytoskeleton to maintain cell shape.

Intravascular blood pumps comprise miniaturized blood pumps capable of being percutaneously or surgically introduced into the vascular system of a patient, typically to provide left and/or right heart support. See, e.g., U.S. Pat. No. 4,625,712 which describes a multiple stage intravascular axial-flow blood pump which can be percutaneously inserted into an artery for heart assist and U.S. Pat. No. 4,846,152 which describes a single-stage intravascular axial flow blood pump, the contents of which are hereby incorporated by reference as if recited in full herein. These blood pumps position the drive unit/motor outside the body (extracorporeal) and use long cable drive systems. The maneuverability and/or durability of these types of blood pumps was often less than desired. During use, components of these devices tended to deteriorate prematurely due to rotational and pulsatile forces experienced by the blood pumps.

Other intravascular blood pumps are configured so that the drive unit/motor and the impeller are directly connected to each other, with the motor and the impeller (pump) housing having the substantially the same outer diameter. See, e.g., U.S. Pat. No. 6,176,848, the contents of which are hereby incorporated by reference as if recited in full herein. While these systems have been used successfully to pump blood, the flow rates provided are typically under 3-4 liters/minute at a counterpressure of about 100 mm Hg. The pumping rate is limited by the low torque limitation of the small “micro” motors.

There is a need for blood pumps that can reduce the foregoing drawbacks of prior art intravascular blood pumps.

### SUMMARY

Embodiments of the present invention provide blood pumps with an extracorporeal motor and a multi-lumen shaft enclosing a long torque cable that is coupled to the motor at one end and to an impeller shaft at another and that provides in-flow fluid path(s) and an out flow (purge) fluid path(s) that cool and/or lubricate the long torque cable.

A distal end portion of the multi-lumen shaft can be in fluid communication with a bearing housing and bearing/bushing held by the bearing housing which are configured to provide a cross-over fluid purge path thereby connecting the in-flow and out-flow fluid paths.

Aspects of the present invention are directed to lubrication/purge fluid path(s).

Embodiments of the invention are directed to a blood pump including an extracorporeal motor housing with a motor coupled to a motor shaft, a torque cable coupled to the motor shaft and extending longitudinally outside of the motor housing, and a multi-lumen shaft having a torque cable lumen sized and configured to encase at least a segment of the torque cable. The torque cable lumen defines at least a portion of an out-flow (purge) fluid path that extends along and about the torque cable. The multi-lumen shaft further includes at least one fluid in-flow lumen in fluid communication with the out-flow (purge) fluid path whereby out-flow fluid in the out-flow path purges debris, cools and/or lubricates the torque cable. The blood pump also includes an impeller shaft attached to the distal end portion of the torque cable. The impeller shaft has an outer surface configured to provide a portion of the out-flow (purge) fluid path with the outer surface configured to be in fluid communication with a distal end portion of the torque cable lumen. The blood pump also includes an impeller coupled to the impeller shaft.

The torque cable lumen can terminate proximally a distance from a distal end of the at least one in-flow lumen of the multi-lumen shaft. A proximal end portion of the impeller shaft can reside adjacent to and/or inside the distal end portion of the torque cable lumen.

The motor shaft can have an axially extending channel sized and configured to receive a portion of the torque cable.

The blood pump can further include a motor cap coupled to the motor and a manifold coupled to a distal portion of the motor cap. The motor cap can have a fluid out-flow port in fluid communication with the torque cable lumen.

The blood pump can further include a manifold surrounding and coupled to a sub-length of the multi-lumen shaft. The manifold can have a fluid in-flow port in fluid communication with the at least one in-flow lumen.

The manifold can be defined by a plurality of cooperating components that are attached together. The manifold can be attached to a proximal end portion of the multi-lumen shaft.

The multi-lumen shaft can include a proximal end portion that resides in the manifold. The proximal end portion of the multi-lumen shaft can have an outer wall with fluid intake ports in fluid communication with the at least one in-flow lumen. The blood pump can further include a fluid in-flow conduit attached to the manifold at one end and to an in-flow fluid source connector at an opposing end.

The blood pump can further include a motor cap enclosing inner and outer seals that sandwich a grease pocket. The motor cap can be coupled to a distal end of the motor and defines a fluid out-flow port that is in fluid communication with the fluid out-flow path of the multi-lumen shaft and provides a part of the fluid out flow path that is external to the inner and outer seals and extends laterally outwardly from the motor cap.

The multi-lumen shaft can be an extruded body that has an outer wall of a first polymer, optionally POLYAMIDE OR POLYIMIDE.

The extruded body can surround a first internal polymer tube, optionally PEBAX, providing the torque cable lumen.

The snorkel tube can be an extruded body encasing a length of a coil.

The motor shaft can be configured to rotate at about 50,000 rpm and have a maximal outer diameter of 0.100 inches thereby reducing surface speed when the shaft is rotating at about 50,000 rpm relative to larger diameters for facilitating a desirable life span of a seal at the distal end portion of the motor.

The blood pump can further include an outlet cage surrounding the impeller and an inlet cage distal of the outlet cage.

The blood pump can further include an impeller shaft that extends distally out of a bearing housing. The impeller shaft can be coupled to a distal end portion of the torque cable.

Optionally, the impeller shaft can extend through a portion of a proximal bushing. A proximal end portion of the impeller shaft and the proximal bushing can both be attached to the torque cable to thereby couple the torque cable to the impeller shaft.

The blood pump can further include a bearing housing that holds a bearing/bushing that surrounds a portion of the impeller shaft. The bearing/bushing can have at least one radially extending open channel that is in fluid communication with a fluid in-flow path provided by the at least one in-flow lumen of the multi-lumen shaft to define a cross-over portion for purge fluid to cross-over from the at least one in-flow lumen to the out-flow (purge) fluid path whereby the bearing/bushing acts as a hydrostatic bearing/bushing.

The blood pump can further include axially spaced apart proximal and distal bushings residing on opposing end portions of the bearing/bushing. The proximal bushing can be attached to the impeller shaft and the torque cable. Optionally, the distal bushing can be attached to the impeller shaft distal to the bearing housing.

The proximal and distal bushings can be configured to define thrush washers whereby the bearing/bushing is trapped therebetween. The proximal and distal bushings can constrain the impeller shaft inside the bearing/bushing while the impeller shaft freely spins in the bearing/bushing.

The multiple lumen shaft can have at least one pressure sensor lumen.

The torque cable can have an axially extending lumen. The axially extending lumen of the torque cable can hold at least a segment of at least one support wire.

The torque cable can have a body of a plurality of overlapping layers of filars arranged in an overlapping pattern defining a cylindrical body.

The bearing housing can hold tube segments forming the at least one in-flow lumen of the multi-lumen shaft and can have lateral passages that fluidly connect fluid from the tube segments across the bearing housing to the bearing/bushing at least one radially extending open channel.

Embodiments of the invention are directed to a method of cooling/lubricating a torque cable of an intravascular blood pump including providing a blood pump with a manifold coupled to a multi-lumen shaft at a proximal end portion and with a bearing housing adjacent a distal end portion of the multi-lumen shaft, proximal to an impeller of the blood pump, the multi-lumen shaft having an in-flow fluid path and an out-flow fluid path, and the out-flow fluid path can extend along and surrounds a torque cable held in the multi-lumen shaft while the torque cable is coupled to an extracorporeal motor; in-flowing a biocompatible fluid into the manifold, then into the in-flow path of the multi-lumen shaft; then flowing the biocompatible fluid from the in-flow path to a cross-over path defined at least in part in a bearing (bushing) surrounding part of an impeller shaft; then out-flowing the biocompatible fluid out of the out-flow path along and about the torque cable thereby purging any debris, cooling and lubricating the torque cable; and pumping blood using the blood pump while in-flowing and out-flowing the biocompatible fluid.

The out-flowing can further include directing some of the biocompatible fluid from the cross-over path to flow over and along a purge path formed in the impeller shaft that is coupled to the impeller, while directing other of the biocompatible fluid continues to the out-flowing along and about the torque cable in the out-flow fluid path.

The out-flowing can further include directing some of the biocompatible fluid from the cross-over path out an impeller shaft/bearing housing interface into a patient body while directing other of the biocompatible fluid continues to the out-flowing along and about the torque cable in the out-flow fluid path.

A portion of the out-flow path can be defined by a torque cable lumen of the multi-lumen shaft, and the torque cable lumen can extend longitudinally.

Aspects of the present invention are directed to a bearing (bushing) for a heart pump.

Embodiments of the invention are directed to a bearing (bushing) for a heart pump that includes a bearing (bushing) body having an axially extending open channel sized and configured to receive an intravascular impeller shaft. The bearing (bushing) body can further include at least one radially extending open channel thereby defining a fluid cross-flow path that is sized and configured to define at least one purge fluid path segment whereby the bearing (bushing) acts as a hydrostatic bearing/bushing.

The bearing (bushing) body can be formed of 3016 PEEK, with carbon fibers in a range of 15-25% by volume and PTFE in a range of 15-25% by volume thereby providing a wear resistant and lubricious body.

The at least one radially extending open channel can have first and second radially extending channel segments that face each other across the axially extending open channel.

The at least one radially extending open channel can have a diameter in a range of about 0.010 inches to about 0.030 inches.

The axially extending open channel can have a diameter in a range of about 0.5 mm to about 1 mm.

The bearing (bushing) can be in combination with an impeller shaft having at least one outer surface segment with a recess defining part of a fluid purge flow path.

Aspects of the present invention are directed to an impeller shaft for a heart pump.

Embodiments of the invention are directed to an impeller shaft sized and configured for an intravascular impeller of a blood pump. The impeller shaft can include a shaft body that extends in an axial direction. The shaft body can have a proximal end portion and a spaced apart opposing distal end portion. The shaft body can have a maximal outer diameter that is in a range of 0.5-1 mm. The shaft body can have an outer surface with at least one recess that extends over at least a portion of a length of the shaft body to thereby define a purge fluid passage.

The at least one recess can define a flat segment on the outer surface.

The at least one recess can be a single recess.

The at least one recess can be a spiral groove.

The flat segment can extend circumferentially a distance of 0.010 mm to 0.025 mm about the outer surface.

The distal end portion can taper in a distal direction from the maximal outer diameter to a reduced diameter. The at least one recess can extend proximal of the distal end portion.

The impeller shaft can have a length in a range of about 10 mm to about 15 mm. The at least one recess can extend a distance that is at least 50% of the length of the shaft body from a proximal end to a location proximal to a distal end of the shaft body.

The at least one recess can have a distal end that terminates a distance in a range of 5-6 mm from a tip of the distal end portion of the impeller shaft.

The shaft body can be metal with a hardness of at least Rockwell C60.

The outer surface of the shaft body can have a smooth finish of RMS 8.

The impeller shaft can further include a diamond like coating (DLC) configured to provide hardness and lubricity.

Aspects of the present invention are directed to an impeller to torque cable assembly for a heart pump.

Embodiments of the invention are directed to an impeller sub-assembly for a blood pump that includes an impeller sized and configured for intravascular placement, an impeller shaft attached to the impeller, a distal bushing arranged to surround and attach to an outer surface of a portion of the impeller shaft and is received in a channel of the impeller and attached to a wall surface of the channel, a proximal bushing arranged to surround and attach to an outer surface of a portion of the impeller shaft at a location proximal to the distal bushing, a bearing (bushing) surrounding a segment of the impeller shaft and residing between the distal and proximal bushings, and a torque cable held inside the proximal bushing, attached to the proximal bushing at a spaced apart location from the impeller shaft.

The sub-assembly can further include a bearing housing surrounding the bearing (bushing).

The bearing (bushing) can have at least one radially extending open channel that is in fluid communication with a fluid in-flow path to define a cross-over portion for purge fluid to cross-over from the fluid in-flow path to an out-flow (purge) fluid path upstream of the bearing/bushing whereby the bearing/bushing also acts as a hydrostatic bearing/bushing.

The proximal bushing can be attached to the impeller shaft and to a torque cable and a support wire.

The proximal and distal bushings can be configured to define thrush washers whereby the bearing/bushing is trapped therebetween. The proximal and distal bushings can constrain the impeller shaft inside the bearing/bushing while the impeller shaft freely spins in the bearing/bushing.

The bearing housing can have an outer surface facing the impeller and can define an impeller/housing interface thereat. The impeller/bearing housing interface can be configured to eject purge fluid toward the impeller.

The torque cable can have a center channel that holds a support wire. The proximal bushing can be crimped onto a distal end portion of the torque cable and support wire at a first location and can be crimped onto the impeller shaft at a second location that is distal to the first location.

The torque cable and support wire can be held in a center lumen of a multiple lumen shaft. The sub-assembly can further include a bearing housing adapter that extends about an outer distal end of the multi-lumen shaft between the bearing housing and the multi-lumen shaft to attach the multi-lumen shaft to the bearing housing.

The bearing (bushing) can have radially extending through channels that define cross-flow passages sized and are configured to connect at least one in-flow lumen of a multi-lumen shaft to an out-flow path provided by a longitudinally extending channel of the bearing (bushing) that is in fluid communication with an out-flow lumen of the multi-lumen shaft.

The impeller shaft can have an outer surface held in a channel of the impeller that defines at least one recess configured to provide a purge fluid passage from purge fluid exiting a bearing housing surrounding the bearing (bushing).

The bearing housing can have an open center channel and at least one longitudinally extending channel residing radially spaced apart from the open center channel and that is in fluid communication with the at least one radially extending open channel of the bearing (bushing).

The at least one radially extending open channel of the bearing housing can be arranged as a first channel and the bearing housing can have first and second radially extending channel segments that face each other across an axially extending open center channel.

The bearing housing can have an external surface that faces the impeller and defines a bearing/impeller interface. A first amount of purge fluid can be configured to exit at the bearing/impeller interface while maintaining a sufficient fluid pressure that prevents blood from entering the bearing housing while a larger second amount of purge fluid is directed back to an out-flow path provided by an out-flow lumen of a multi-lumen shaft.

The multi-lumen shaft can have a center lumen that extends longitudinally and defines at least part of the out-flow path. The multi-lumen shaft can have at least one in-flow lumen defining at least part of an in-flow path.

The at least one in-flow lumen can extend circumferentially about the center lumen.

The at least one in-flow lumen can be provided by first and second polymer tubes held at least partially in an extruded body of the multi-lumen shaft.

Aspects of the present invention are directed to a motor assembly with seals for a blood pump.

Embodiments of the invention are directed to a motor assembly for a blood pump that includes a motor housing, a motor in the motor housing, a motor shaft coupled to the motor, a torque cable coupled to the motor shaft, and inner and outer seals that are coupled together and sandwich a grease pocket. The motor shaft with the torque cable therein can extend through the inner and outer seals. The torque cable can be sized and configured to a distance outside the motor shaft and outside of the motor housing.

The inner seal can be configured as a memory lip seal and the outer seal can be configured as a cooperating a stainless-steel spring that applies pressure to the torque shaft. The grease pocket can include food grade grease.

The motor can include a motor end cap that defines a fluid out-flow port that is in fluid communication with a fluid outflow path of a multi-lumen shaft that encloses the torque cable and that provides at least in part, a fluid out-flow path that is external to the inner and outer seals and that extends laterally outwardly from the motor cap to the fluid out-flow port.

The motor assembly can further include a manifold coupled to the motor end cap. The multi-lumen shaft can extend through the manifold and the manifold can include an in-flow port that is in fluid communication with at least one in-flow lumen provided by the multi-lumen shaft.

Aspects of the present invention are directed to a motor shaft for a blood pump.

Embodiments of the invention are directed to a motor shaft for a blood pump that includes a cylindrical metal body with a center channel that is open and sized and configured to receive a flexible torque cable. The cylindrical body can have a maximal outer diameter that is in a range of 0.050 inches to 0.100 inches and can have an outer surface having a diamond like coating (DLC) to provide hardness and lubricity.

Aspects of the present invention are directed to a support wire and torque cable for a blood pump.

Embodiments of the invention are directed to a blood pump including an extracorporeal motor housing with a motor coupled to a motor shaft. The motor shaft can have an axially extending channel. The blood pump can further include a torque cable coupled to the motor shaft and extending longitudinally a distance outside the blood pump housing, a multi-lumen shaft having a torque cable lumen sized and configured to encase a segment of the torque cable and define at least a portion of a purge fluid out-flow path. The multi-lumen shaft can further include at least one in-flow lumen. The blood pump can further include an impeller coupled to a distal end of the torque cable and a support wire that extends through at least a segment of the axially extending channel of the torque cable.

The support wire can extend through the motor shaft and extends proximally to terminate in and/or to extend out of a proximal end of the torque cable.

The torque cable can extend through an entire length of the motor shaft.

The torque cable can be configured to float inside the torque cable lumen for at least a major length thereof.

The support wire can terminate a distance in a range of 1-3 inches distal to a manifold coupled to the motor. The manifold can define a portion of a fluid in-flow path that is in fluid communication with the at least one in-flow lumen.

The support wire can be a first support wire. The blood pump can further include a second support wire that extends through the torque cable, distally of the first support wire.

The first support wire and the second support wire can be Nitinol support wires.

The torque cable can have a body of a plurality of overlapping layers of filars arranged in an overlapping pattern defining a cylindrical body with the axially extending lumen centrally arranged to extend therethrough.

Different layers can provide respective one or more filars at different angles so that a filar(s) of a first layer intersects a filar(s) on one or more adjacent layers.

The blood pump can further include a motor cap coupled to the motor and a manifold coupled to the motor cap. The motor cap can have a fluid out-flow port in fluid communication with the out-flow path.

The blood pump can further include a manifold coupled to the motor cap and include a fluid in-flow port in fluid communication with the at least one in-flow lumen. The manifold can surround a sub-length of the multi-lumen shaft.

The manifold can be defined by a plurality of cooperating components that are attached to a proximal end portion of the multi-lumen shaft.

The multi-lumen shaft can have a proximal end portion that resides in the manifold. The proximal end portion of the multi-lumen shaft can have an outer wall with fluid intake ports in fluid communication with the at least one in-flow lumen. The blood pump can further include a fluid in-flow conduit attached to the manifold at one end and to a fluid in-flow connector at an opposing end.

The blood pump can further include a motor cap enclosing inner and outer seals that sandwich a grease pocket. The motor cap can define an end cap of the motor housing and provide a fluid out-flow port that is in fluid communication with a fluid out-flow path of the multi-lumen shaft. The motor cap can define a part of an out-flow path of the blood pump that is external to the inner and outer seals and that can extend laterally outwardly from the motor cap.

The multi-lumen shaft can have an extruded body that has an outer wall of a first polymer, optionally POLYAMIDE or POLYIMIDE.

The extruded body can surround a first internal polymer tube, optionally PEBAX, providing the torque cable lumen, and can provide the at least one in-flow lumen, or the extruded body can be provided by a first extruded body providing the torque cable lumen and a second cooperating extruded body providing a coaxially arranged in-flow lumen.

The motor shaft can be configured to rotate at about 50,000 rpm and have a maximal outer diameter of 0.100 inches over at least a major length thereof thereby reducing surface speed when the shaft is rotating at about 50,000 rpm relative to larger diameters for facilitating a desirable life span of a seal at the distal end portion of the motor shaft.

The blood pump can further include an out-flow cage surrounding the impeller and an inlet cage distal of the outlet cage.

The multi-lumen shaft can have a distal end portion that terminates at a location that is spaced apart from and proximal to the impeller.

The blood pump can further include an impeller shaft that extends distal out of a bearing housing.

The blood pump can further include an impeller shaft that has at least one exterior surface segment that defines a portion of the (purge) out-flow fluid path.

The blood pump can further include a bearing housing that holds a bearing (bushing) that surrounds a segment of the impeller shaft. The bearing (bushing) can have at least one radially extending channel that is in fluid communication with a fluid in-flow path provided at least in part by the at least one in-flow lumen of the multi-lumen shaft to define at least one cross-over (purge) fluid path whereby the bearing (bushing) acts as a hydrostatic bearing/bushing.

The blood pump can further include axially spaced apart proximal and distal bushings residing on opposing sides of the bearing/bushing. The proximal bushing can be attached to the impeller shaft, the torque cable and a second support wire. The distal bushing can be attached to the impeller shaft outside the bearing housing.

The proximal and distal bushings can be configured to define thrush washers whereby the bearing/bushing is trapped therebetween.

The multiple lumen shaft can include at least one pressure sensor lumen.

The blood pump can further include an impeller shaft coupled to the impeller and to the torque cable. The impeller shaft can define a portion of the (purge) fluid out-flow path in fluid communication with a cross-over fluid path provided by a bearing/bushing that surrounds a portion of the impeller shaft.

The bearing/bushing can surround a portion of an impeller shaft that is coupled to the impeller. Biocompatible purge fluid from the at least one in-flow lumen can travel distally through holes in the bearing/bushing providing the cross-over fluid path then along a portion of the impeller shaft into and along the torque cable lumen thereby purging debris and/or cooling and lubricating the torque cable.

The bearing housing can have an external surface that faces the impeller and defines a bearing/impeller interface. A first amount of purge fluid can be configured to exit at the bearing/impeller interface into a patient while maintaining a sufficient fluid pressure that prevents blood from entering the bearing housing while a greater amount of purge fluid is configured to exit the blood pump along an outflow path provided by the multi-lumen shaft.

The blood pump can be configured to pump blood through the outlet cage at a rate in a range of 2-7 liters/minute over at least 6 days of continuous intravascular use while continuously providing biocompatible fluid to the at least one in-flow lumen, then to the out-flow path.

Aspects of the present invention are directed to a motor cap for a blood pump.

Embodiments of the invention are directed to a motor cap for a blood pump including a motor cap body having a chamber sized and configured to hold inner and outer cooperating seals, a neck configured to receive a neck of a manifold, and a proximal, longitudinally extending wall with threads configured to couple to a motor. The end cap can have a fluid out-flow port.

The fluid out-flow port can extend laterally outward from the longitudinally extending wall.

The neck can have an open channel that is sized and configured to receive a torque cable.

The chamber can have an inner surface that defines a portion of a purge fluid out flow path of the blood pump from a multi-lumen shaft to the fluid out-flow port.

Aspects of the present invention are directed to a manifold for a blood pump.

Embodiments of the invention are directed to a manifold for a blood pump including a first member having axially spaced apart proximal and distal ends with an outer wall with a fluid in-flow port extending therethrough to an open internal manifold chamber, a second member having a neck and attached to the proximal end of the first member, the neck having an open through channel and sized and configured to couple to an end cap of a motor housing, and a third member attached to the distal end of the first member and having an open through channel, wherein the open through channel of the third member has a larger diameter than the open through channel in the second member.

The manifold can further include a multi-channel lumen enclosing a torque cable having a proximal end portion that is inside the manifold and attached to the first member and/or the second member with the torque cable extending outside the second member and with the multi-lumen shaft extending outside the third member.

Aspects of the present invention are directed to methods of assembly and fabrication of a blood pump.

Embodiments of the invention are directed to a method of assembling and/or fabricating a blood pump including providing a multi-lumen shaft having a torque cable lumen and an in-flow lumen. The multi-lumen shaft can be flexible and elongate and configured for intra-arterial tortuous path insertion to a heart of a patient. The method can further include attaching a proximal end portion of the multi-lumen shaft to a manifold, inserting a proximal end portion of a torque cable that extends a distance outside the multi-lumen shaft to a motor shaft of a motor, before or after the attachment, attaching a proximal bushing to an impeller shaft and the torque cable, and attaching an impeller to the impeller shaft.

The method can further include, before inserting the proximal end portion of the torque cable to the motor shaft, inserting the proximal end portion of the torque cable through an end cap in line with the motor shaft.

The method can further include attaching a bearing housing adapter to a bearing housing and outer wall of the multi-lumen shaft.

The step of attaching the impeller can include attaching a distal bushing to the impeller shaft and inserting the impeller shaft with the distal bushing into a channel in the impeller, then attaching the distal bushing and impeller shaft to the impeller.

The method can further include, optionally before attaching the impeller shaft to the impeller, attaching a bearing/bushing to a bearing housing, and inserting the impeller shaft through the bearing/bushing.

Further features, advantages and details of the present invention will be appreciated by those of ordinary skill in the art from a reading of the figures and the detailed description of the preferred embodiments that follow, such description being merely illustrative of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF DRAWINGS

FIG. 1 is a side view of a blood pump, with one part of a shell handle omitted to reveal internal components, according to embodiments of the present invention.

FIG. 2 is another side view of the blood pump shown in FIG. 1, rotated at 90 degrees from the orientation shown in FIG. 1.

FIG. 3 is a greatly enlarged, section view of a proximal end portion of the blood pump shown in FIG. 1.

FIG. 4A is a greatly enlarged lateral section view of a multi-lumen shaft of the device shown in FIG. 1.

FIG. 4B is a greatly enlarged lateral section view of another embodiment of a multi-lumen shaft for a blood pump according to embodiments of the present invention.

FIG. 5 is an enlarged section view of an impeller assembly portion of the blood pump device shown in FIG. 1.

FIG. 6 is a greatly enlarged section view of a portion of the blood pump device shown in FIG. 1 illustrating example in-flow and out-flow fluid paths according to embodiments o the present invention.

FIG. 7 is a schematic illustration of an example use of the blood pump device shown in FIG. 1 showing the inlet cage inside the left ventricle and the outlet cage outside the aorta according to embodiments of the present invention.

FIG. 8A is a side perspective view of an example bearing housing for the blood pump shown in FIG. 3 according to embodiments of the present invention.

FIGS. 8B and 8C are end views of the example bearing housing shown in FIG. 8A.

FIG. 9A is a side view of the bearing housing shown in FIG. 9A.

FIG. 9B is a section view taken along line 9B-9B in FIG. 9A.

FIG. 9C is another side view of the bearing housing shown in FIG. 9A, rotated 90 degrees from the orientation shown in FIG. 9A.

FIG. 10A is an end view of an example bearing (bushing) shown in FIG. 3 according to embodiments of the present invention.

FIG. 10B is a side view of the bearing (bushing) shown in FIG. 10A.

FIG. 11 is a schematic illustration of an example torque cable according to embodiments of the present invention.

FIG. 12A is a side perspective view of an example impeller shaft according to embodiments of the present invention.

FIG. 12B is a side view of the impeller shaft shown in FIG. 12A.

FIG. 12C is a side perspective view of another example impeller shaft according to embodiments of the present invention.

FIG. 13 is another side view of the impeller shaft shown in FIG. 12A, rotated 90 degrees from the orientation shown in FIG. 12A.

FIG. 14A is a section view of the device shown in FIG. 12A, taken along line 14A-14A in FIG. 13.

FIG. 14B is a section view of the device shown in FIG. 12A, taken along line 14B-14B in FIG. 13.

FIG. 15 is an enlarged sectional view of cooperating inner and outer seals for the motor assembly for the blood pump shown in FIG. 3 according to embodiments of the present invention.

FIG. 16 is an enlarged (2.5:1) view of the impeller region of the blood pump shown in FIG. 1 illustrating example biocompatible (purge) fluid flow directions according to embodiments of the present invention.

FIG. 17 is an enlarged partial section view of a portion of a proximal segment of the blood pump illustrating example biocompatible (purge) fluid flow directions according to embodiments of the present invention.

FIG. 18 is a flow chart of example actions that can be used to operate a blood pump according to embodiments of the present invention.

FIG. 19A is a front view of an example blood pump according to embodiments of the present invention.

FIG. 19B is a greatly enlarged view of a distal end portion of the blood pump shown in FIG. 19A.

FIG. 20 is a greatly enlarged view of a portion of another embodiment of a catheter blood pump showing an expandable impeller blade configuration in an expanded configuration and in the heart of a patient according to embodiments of the present invention.

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. The abbreviation “FIG.” may be used interchangeably with “Fig.” and the word “Figure” in the specification and figures. It will be appreciated that although discussed with respect to a certain embodiment, features or operation of one embodiment can apply to others.

In the drawings, the thickness of lines, layers, features, components and/or regions may be exaggerated for clarity and broken lines (such as those shown in circuit of flow diagrams) illustrate optional features or operations, unless specified otherwise. In addition, the sequence of operations (or steps) is not limited to the order presented in the claims unless specifically indicated otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when a feature, such as a layer, region or substrate, is referred to as being “on” another feature or element, it can be directly on the other element or intervening elements may also be present. In contrast, when an element is referred to as being “directly on” another feature or element, there are no intervening elements present. It will also be understood that, when a feature or element is referred to as being “connected” or “coupled” to another feature or element, it can be directly connected to the other element or intervening elements may be present. In contrast, when a feature or element is referred to as being “directly connected” or “directly coupled” to another element, there are no intervening elements present. Although described or shown with respect to one embodiment, the features so described or shown can apply to other embodiments. The term “about” means that the noted number can vary by +/−20%.

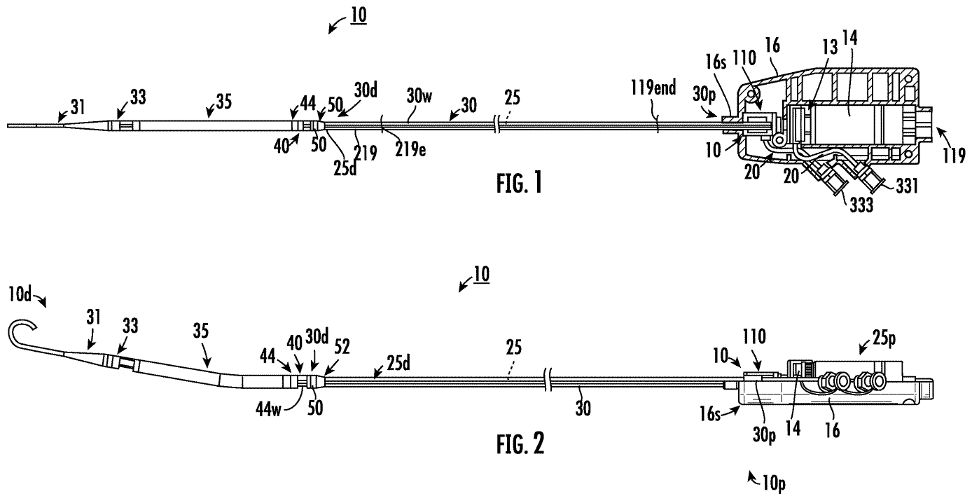

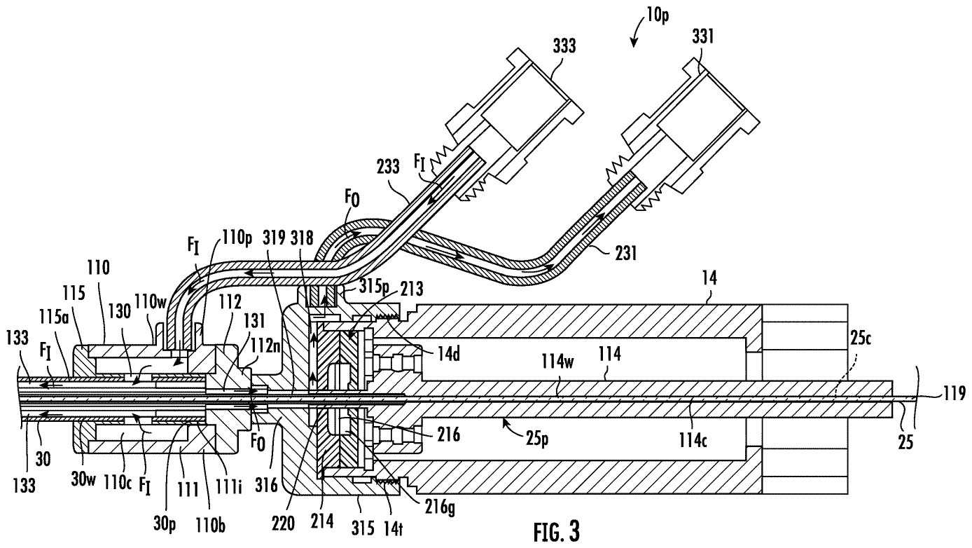

Referring to FIGS. 1 and 2 an example blood pump 10 is shown. The blood pump 10 comprises a motor 14, a multi-lumen shaft 30 that encloses a torque cable 25, an inlet cage 33 (blood intake), a (snorkel) tube 35 extending between the inlet cage 33 and an impeller 40, and an outlet cage 44 (pumped blood outlet) with blood outlet windows 44w. A snorkel 31 can be attached to the snorkel tube 35 and be positioned at a distal end 10d of the blood pump 10. The snorkel/snorkel tube may be provided in a number of configurations.

The blood pump 10 can also have a manifold 110 that is coupled to the motor 14. The manifold 110 has a manifold chamber 110c. As will be discussed further below, with respect to FIG. 3, the manifold 110 can sealably enclose a sub-length of the shaft 30, typically at least a segment of the proximal end portion 30p of the multi-lumen shaft 30 and can define at least a portion of a (purge) fluid in-flow path into at least one aperture/port 130 in an outer wall 30w of the multi-lumen shaft 30, then into at least one in-flow lumen(s) 133 provided by the multi-lumen shaft 30. The term “in-flow” can be used interchangeably with the term “inflow” herein. The term “out-flow” can be used interchangeably with the term “outflow” herein.

The blood pump 10 can also have a bearing housing 50 adjacent the impeller 40 with a bearing housing adapter 52 that couples an outer wall 30w of the multi-lumen shaft 30 to the bearing housing 50. As will be discussed below, the bearing housing 50 can comprise a lateral cross-flow passage 55 that is in fluid communication with an a radially extending passage 602 of a the bearing/bushing 600 and a longitudinal channel 605 thereof, that provides part of the out-flow path Fo.

The multi-lumen shaft 30 has a proximal end portion 30p that is adjacent the motor 14 and an opposing distal end portion 30d that terminates adjacent the impeller 40. The torque cable 25 also has a proximal end portion 25p that is adjacent the motor 14 and an opposing distal end portion 25d that terminates adjacent the impeller 40. The torque cable 25 can also be interchangeably referred to as a “drive cable”. The torque cable 25 can be directly or indirectly attached to the impeller 40 at the distal end portion 25d of the torque (drive) cable 25 and to the motor 14 at the proximal end portion 25p of the torque (drive) cable 25.

The motor 14 can be held in a motor housing 16. The motor housing 16 can be provided as a cooperating pair of handle shells 16s. The motor housing 16 can be an extracorporeal housing.

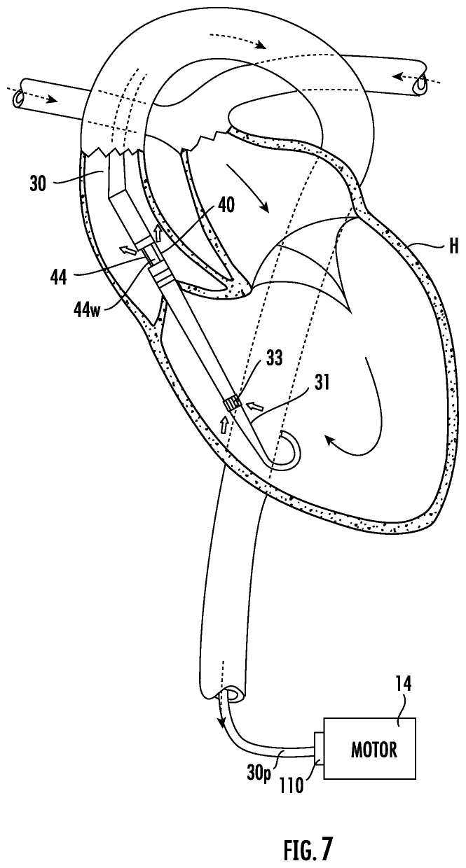

An intrabody portion of the blood pump 10 (distal to the housing 16) is configured to be inserted into the aorta from a remote entry point, such as an incision below the groin that provides access into a femoral artery. The intrabody portion of the blood pump 10 (snorkel 31 leading the way), then passes through the descending aorta until it reaches the ascending aorta, near the heart. The multi-lumen shaft 30 encloses the torque cable 25 and can have a length sufficient to position the motor 14 to be extracorporeal. The proximal end portion 30p of the multi-lumen shaft 30 can reside outside the body, typically near the patient's groin, at an end portion opposing the impeller 40 and snorkel 31. FIG. 7 illustrates an example operational configuration in the heart H with the blood intake cage 33 and snorkel 31 in a left ventricle (LV) of a heart of a patient, the impeller 40 and blood outlet cage 44 positioned in the aorta, proximate the coronary arteries, above the aortic valve to discharge pumped blood out of cage windows 44w and into the aorta, while the motor 14 and motor housing 16 are outside the patient. The arrows indicate primary blood flow into the intake cage 33 and out of the outflow cage 44 during operation.

Generally stated, when the proximal end portion of the torque cable 25 is mechanically rotated by a motor shaft 114 of the motor 14, typically located outside the patient's body, it conveys the rotational force through the length of the multi-lumen shaft 30, causing the impeller 40 to spin at high speed near the heart.

The blood pump 10 can be particularly suitable in providing ventricular assist during surgery or providing temporary bridging support to help a patient survive a crisis.

The motor 14 is arranged to drive the torque cable 25 in the multi-lumen shaft 30 which in turn drives the impeller 40/pump unit. The motor 14, being operated at an extracorporeal site, can have any desired size. The multi-lumen shaft 30 provides continuous lubrication by a biocompatible (purge) liquid. A part of this liquid can exit through a bearing housing/impeller shaft interface and thus enter the blood stream. The remaining part can be directed to flow through an out-flow path and be collected extracorporeally after passing through a lumen 131 provided in the multi-lumen shaft 30 that holds the drive cable 25.

The multi-lumen shaft 30 and the impeller 40 may be dimensioned to any suitable diameter for intravascular applications. For example, the range of sizes may include, but is not necessarily limited to, 9 French to 30 French, although the range is typically in a range of 14 French to 24 French, and more typically in a range of 18 French to 20 French.

The blood pump 10 can comprise first and second support wires 119, 219 that are longitudinally spaced apart and reside inside the torque cable 25. Referring to FIG. 1, the first support wire 119 can have a distal end 119e that terminates a range of 1-3 inches from the manifold 110 and extends at least partially through the motor shaft 114, shown as extending entirely through the motor shaft in FIG. 3. The second support wire 219 can have a proximal end 219e that terminates a range of 1-3 inches from the proximal end of the impeller shaft 140. The first support wire 119 can support the torque cable 25 at a high torque area (at the motor 14) so that the torque cable 25 does not collapse under load. The first support wire 119 can also act as a strain relief when it exits a distal end of the manifold 110. The second support wire 219 can allow the impeller shaft 140 and torque cable 25 to be crimped together by using the proximal bushing 400 without collapsing the (hollow) torque cable 25. The second support wire 219 can also act as a strain relief.

In some embodiments, the first and second support wires 119, 219 can be provided as a single support wire instead of separate support wires and the single support wire may extend substantially an entire length of the torque cable 25 or reside only at a proximal end portion or only at a distal end portion of the torque cable 25.

Turning now to FIG. 3, an enlarged section view of the proximal end portion 10p of the blood pump 10 is shown. The motor 14 has a motor shaft 114 that can have a through channel 114c that holds a proximal portion 25p of the torque cable 25. The torque cable 25 can extend distally out of the channel 114c of the motor shaft 114 into a lumen 131 of the multi-lumen shaft 30. The torque cable 25 can be bonded to the inner wall 114w of the channel 114c. The torque cable 25 can extend through at least 50% of an axially extending length of the channel 114c. The torque cable 25 can extend entirely through the channel 114c with a greater length in a distal direction outside the motor 14 facing the impeller 40 than in a proximal direction outside the motor 14. The motor shaft 114 can be metal and may have a diamond like coating (DLC) on an inner and/or outer surface thereof to provide hardness, improved surface finish and lubricity. The outer diameter of the motor shaft 114 is preferred to be as small as possible to reduce the surface speed which improves the lifespan of the seal. In some embodiments, the surface speed is about 773 ft/min when the motor shaft 114 is rotating at about 50,000 rpm. The maximal outer diameter of the motor shaft 114 over at least a major portion of its length (50% or greater) can be in a range of 0.0100 inches to 0.050 inches, such as about 0.060 inches.

The lumen 131 that holds the torque cable 25 can be described as a “torque cable lumen.” The torque cable lumen 131 can define at least part of a purge liquid out-flow path Fo, shown by arrows pointing toward the outflow conduit 231 and outflow connector 331.

The blood pump 10 can also include at least one inflow conduit 233 and inflow connector 333 that connects to a pressurized biocompatible fluid source, such as saline or saline mixture, a medical (biocompatible) grade lubricant, glucose solution and the like. The connectors 331, 333 can be luer connectors.

The inflow conduit 233 can be in fluid communication with the manifold 110 and at least one inflow lumen 133 of the multi-lumen shaft 30 that can define at least part of a (purge) fluid inflow path F_{I}, shown by arrows that point toward the distal end portion 30d of the shaft 30 and distal end portion of the manifold 110.

Referring to FIGS. 3 and 15, the motor 14 can comprise cooperating inner and outer seals 213, 214, which can sandwich a pocket 216 comprising grease 216g therebetween. The torque cable 25 can extend through a channel 220 formed through the pocket 216 and inner and outer seals 213, 214, respectively. The grease 216g can be a food grade grease, such as silicone grease.

The inner and outer seals 213, 214, respectively can cooperate to inhibit or prevent the fluid out-flow from entering the motor 14. The inner and outer seals 213, 214, respectively can be formed of different materials. One of the seals 213, 214 can comprise a high performance filled UHMWPE material such as UPC25 from BALSEAL, Foothill Ranch, CA, USA. The UPC25 is a high temperature resistant filled UHMWPE material designed to perform in chemical and thermal cycling applications with temperatures ranging up to 135 deg. Celsius and is well-suited for rotary and reciprocating applications. The other seal of the seals 213, 214 can comprise a polyimide-filled PTFE compound such as SP191 from BALSEAL, Foothill Ranch, CA, USA. SP191 exhibits low friction and minimal wear in rotary and reciprocating sealing applications and is FDA compliant and is low-outgassing per ASTM E595: see Material Data Sheet M-64 (Rev. 02:08-18-20). The outer seal 214 can comprise a 316SL stainless steel spring that applies sealing pressure to the motor shaft 114. The distal/outer seal 214 can be configured as a flange seal and can comprise the UPC25 material and with the 316L SS spring. The inner/proximal seal 213 can be configured as a memory lip seal and can comprise the SP191 material.

Still referring to FIG. 3, the manifold 110 can be provided as a three-piece body 110b, with the three pieces coupled together, optionally bonded together. The three pieces can include a first end cap 112 and an opposing second end cap 115 coupled to a tubular (primary) body 111. The tubular body 111 can define a chamber 110c that extends about the multi-lumen shaft 30. The tubular primary body 111 can have an outer wall 110w with at least one fluid port 110p that couples to an end portion of the conduit 233. The multi-lumen shaft 30 can comprise an outer wall 30w with at least one aperture 130 defining an in-flow port so that fluid in-flows from the in-flow conduit 233 to the manifold 110, then into the in-flow lumen 133 and longitudinally to the distal end portion 30d of the multi-lumen shaft 30.

The first end cap 112 can hold an end of the proximal portion of the multi-lumen shaft 30. The tubular body 111 can have an inner wall that steps in to hold an outer wall 30w of the multi-lumen shaft 30. The first end cap 112 can be bonded or otherwise attached to the inner wall 111i of the tubular body 111. The second end cap 115 can be bonded or otherwise attach to an opposing end of the tubular body 111. The second end cap 115 has an aperture 115a that is sized and configured to allow the multi-lumen shaft 30 to extend out of the manifold 110. The second end cap 115 can be bonded and/or sealed to the outer wall 30w of the multi-lumen shaft 30.

The manifold 110 (shown as via the first end cap 112) can have a neck 112n that projects outward and couples to a motor cap 315 that has a chamber that receives and resides about the inner and outer seals 213, 214, respectively. The motor cap 315 can have a longitudinally projecting neck 316 that faces and couples to the neck 112n of the first end cap 112. The motor cap 315 can threadably attach to threads 14t at a distal end 14d of the motor 14.

The motor cap 315 can provide at least one port 315p that couples to the conduit 231 to define a part of the out-flow path Fo. The motor cap 315 can also define a portion of the outflow path Fo that extends from the manifold 110 to the conduit 231 via an internal fluid interface 318 that can extend longitudinally along a short channel 319 holding the torque cable 25, then radially across a portion of a diameter of the motor cap 315 between an outer surface of the outer seal 214 and an inner surface of the motor cap 315 thereat.

In some embodiments, the torque cable 25 can have an open center channel 25c that can receive a support wire 119. The support wire 119 can be NITINOL and may have a diameter in a range of 0.010 inches and 0.015 inches, and in some particular embodiments, can be about 0.012 inches.

Referring to FIG. 11, the torque cable 25 can be formed with wound filars 26. The filars 26 can be wound to form a cylindrical body of a plurality of overlapping layers of filars arranged in layers arranging the filars in an overlapping pattern at different angles at each adjacent layer with the axially extending lumen/channel 25c centrally arranged to extend therethrough. Different layers can provide respective one or more filars at different angles so that filars at one layer intersect filars of an adjacent layer(s). The filars 26 can be thin metal wires, such as stainless steel or other suitable material, and the cylindrical body can be sufficiently flexible to resiliently bend in at least two dimensions. In some embodiments, a plurality of filars 26 can be used to define a single strand and a plurality of strands of the filars 26 can then be wound or twisted together to form the torque cable 25. In some embodiments, eight filars 26 can define a respective strand and eight strands can be wound together to form the torque cable 25. Suitable filars 26 for the torque cable 25 can be obtained from Asahi Intecc USA or Asahi Intecc Global.

In some embodiments, the torque cable 25 can have an open center channel 25c along at least part of its length. The support wire 119 may be free-floating in the center channel 25c or be frictionally or adhesively attached at one or more locations. The support wire 119 can facilitate assembly of some components and/or provide strain relief and/or anti-kink support to the flexible torque cable 25 and/or multi-lumen shaft 30.

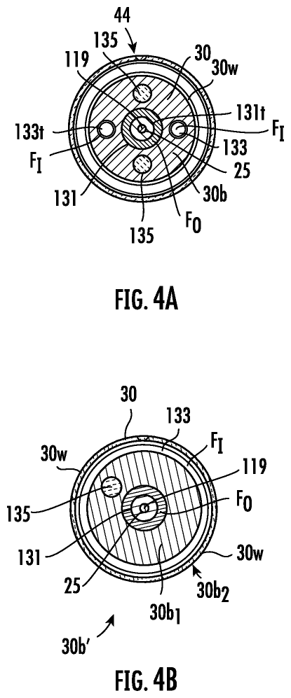

FIG. 4A illustrates an example multi-lumen shaft 30 with a plurality of internal lumens 131, 133, 135. In this view, the (blood) outflow cage 44 is also shown, but it is not part of the body 30b of the multi-lumen shaft 30. The body 30b of the multi-lumen shaft 30 can be provided as an extruded body 30b with multiple (substantially parallel) longitudinally extending lumens 131, 133 and the one or more ports 130 extending through the outer wall 30w (FIG. 3) to the in-flow lumen(s) 133. The body 30b can be an extruded body of polyamide or polyimide.

A separate tube 131t, such as a PEBAX tube, can be used to provide the lumen 131 that encases the torque cable 25 and provide at least a portion of the (fluid purge) outflow Fo path. Alternatively, the lumen 131 can be directly formed in the body 30b of the multi-lumen shaft 30. The at least one in-flow lumen 133 can be provided as a pair of diametrically opposed lumens as shown. The at least one in-flow lumen(s) 133 can be provided as a plurality of separate tubes or passages directly formed in the multi-lumen shaft body 30b. The at least one in-flow lumen 133 can be provided as polymer tubes (optionally polyimide tubes) 133t.

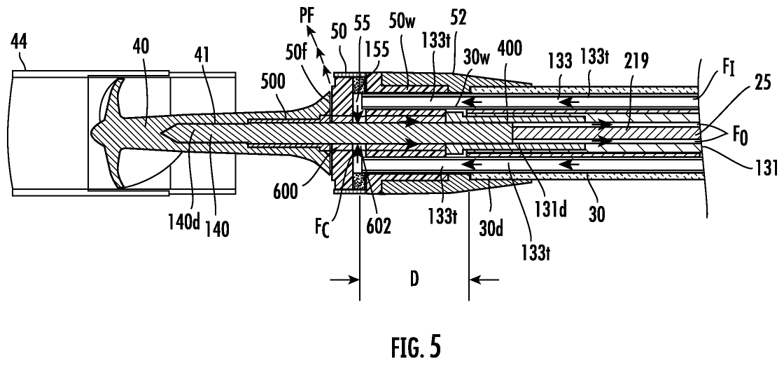

Referring to FIG. 5, the at least one in-flow lumen 133 can extend a distance “D” distally of a distal end 131d of the torque cable lumen 131. Stated differently, the torque cable lumen 131 can terminate proximally a distance from a distal end of the inflow lumen 133. The distance “D” can be in a range of 10-30% of a length of the impeller shaft 140. The distance D can be in a range of 0.1 mm to 0.3 mm, in some embodiments. The impeller shaft 140 can define a portion of the outflow path Fo extending between the cross-over Fc purge fluid flow path to the distal end 131d of the torque cable lumen 131. The impeller shaft 140 and configurations of the inflow and outflow lumens 133, 131, respectively can provide structural rigidity/durability to the multi-lumen shaft 30.

The multi-lumen shaft 30 can also include at least one pressure sensor channel 135, shown as two diametrically opposed pressure sensor channels configured to hold (optical) pressure sensors. The at least one pressure sensor channel 135 can be circumferentially spaced apart from the in-flow lumens 133 and be radially aligned (at a common radius) with the in-flow lumens 133. Other pressure sensors and arrangements can be used.

FIG. 4B illustrates another example extruded body 30b with an in-flow lumen 133 that is provided as an outer ring surrounding the out-flow lumen 131, arranged to provide concentric or coaxial lumen configurations.

The multi-lumen shaft 30 can be provided in a number of ways. For example, the multi-lumen shaft 30 can comprise an extrusion/extruded body 30b with multiple lumens. The center lumen 131 can be a different material than the in-flow lumens 133. This configuration can be provided by a co-extrusion of separate materials extruded at the same time. In other embodiments, such as a coaxial embodiment per FIG. 4B, there can be two separate “tube” extruded bodies 30b1, 30b2, forming the coaxially arranged in-flow and out-flow lumens, of the same or different materials, but the two extruded tube bodies are coaxially positioned, one surrounding the other and can be extruded separately and then assembled together.

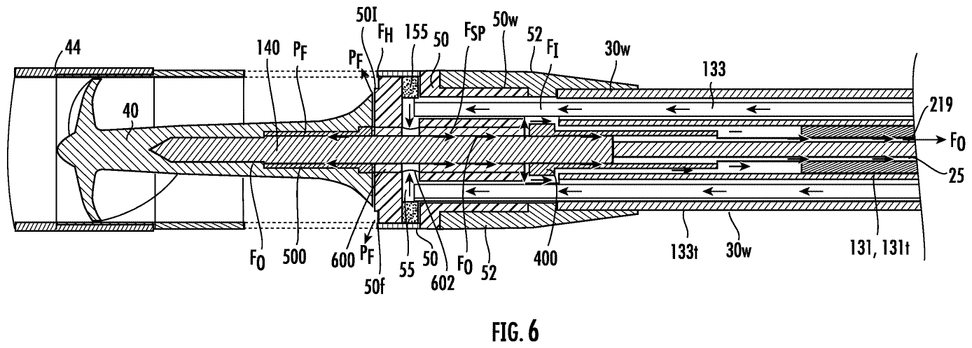

Referring to FIGS. 5 and 6, enlarged section views of a portion of the blood pump 10 that is adjacent the impeller 40. As shown, the impeller 40 is attached to an impeller shaft 140 that extends out of a distal end portion 30d of the multi-lumen shaft 30. A proximal bushing 400 and a distal bushing 500 can sandwich an intermediate bushing 600 (sometimes referred to as a “bearing” but functionally, this component does not have “bearings” or movable components that rotate so that “bushing” may be a more accurate term). Also, for ease of discussion in light of the proximal and distal bushings, the term “bearing/bushing”, “bearing (bushing)”, or “bushing/bearing” and the like 600 can be used herein interchangeably to refer to the bushing 600 component.

The bearing/bushing 600 can be formed of a polymer such as PEEK. In some embodiments, the bearing/bushing 600 can be formed of 3016 PEEK, with carbon fibers in a range of 15-25% (by volume carbon fiber and PTFE in a range of 15-25% (by volume) thereby providing a wear resistant and lubricious body.

A distal end 25d of the torque cable 25 can be coupled to the proximal bushing 400 which can be attached to the impeller shaft 140. A distal end portion 140d of the impeller shaft 140 can be inserted into a channel 41 of the impeller 40. The distal bushing 500 can be crimped to the impeller shaft 140. The impeller 40 can be press fit and/or bonded to the impeller shaft 140 and distal bushing 500. The proximal bushing 400, which can act as a thrust washer, can be placed over a proximal end portion 140p of the impeller shaft 140. The proximal bushing 400 can be attached, e.g., crimped and/or bonded, onto the impeller shaft 140 and torque cable 25, and second support wire 219, where used. The proximal bushing 400 then defines part of an assembly that can be attached, such as press-fitted and/or bonded, to the bearing housing 50.

The bearing/bushing 600 can be press-fit into the bearing housing 50 and no adhesive is required. However, the bearing/bushing 600 can be bonded to the bearing housing 50 in some embodiments.

The distal and proximal bushings 500, 400, respectively, can act as thrust washers “trapping” the bearing/bushing 600. The thrust washers/bushings 400, 500 can constrain the impeller shaft 140 inside the bearing/bushing 600, while allowing the impeller shaft 140 to freely spin in the bearing/bushing 600. The distal and proximal bushings 500, 400 can hold the impeller shaft 140 in position to maintain the clearance gap providing the impeller 40 and bearing housing interface 50I providing a bearing housing outflow path F_{H} of purge fluid at this interface 50I.

A second support wire 219 can extend through the torque cable 25 in the region adjacent the impeller shaft 140 and can extend proximally from the proximal end of the impeller shaft 140 a range of 1-3 inches, in some embodiments.

The bearing/bushing 600 can reside in the bearing housing 50. The bearing housing 50 can define an impeller/interface 50I between an outer surface 50f of the bearing housing 50 and the impeller 40. The bearing outer surface 50f that faces the proximal end 40p of the impeller 40 and is closely spaced apart a distance from the proximal end 40p of the impeller 40. The distance can be in a range of about 0.001 inch to about 0.015 inches. The bearing housing 50 cooperates with the bearing/bushing 600, the impeller shaft 140 and the multi-lumen shaft 30 to define a purge fluid path P_{F} at the interface 50 that directs fluid to exit at the face 50f radially spaced apart from the proximal end 40p of the impeller 40. The pressure/flow rate of the purge fluid P_{F} at this impeller/bearing housing interface is relatively low and able to be ejected into blood but sufficient to keep blood from entering the bearing/bushing 600 and/or the bearing housing 50.

A bearing housing adapter 52 can attach an outer wall 30w of the multi-lumen shaft 30 to an outer wall 50w of the bearing housing 50. The bearing housing adapter 52 can taper inward from an outer diameter of the bearing housing 50 in a direction toward the motor 14.

Referring to FIG. 6, a portion of the proximal bushing 400 and the proximal end of the impeller shaft 140 can reside inside the out-flow lumen 131 and/or inside the out-flow tube 131t, in-line with the torque cable 25.

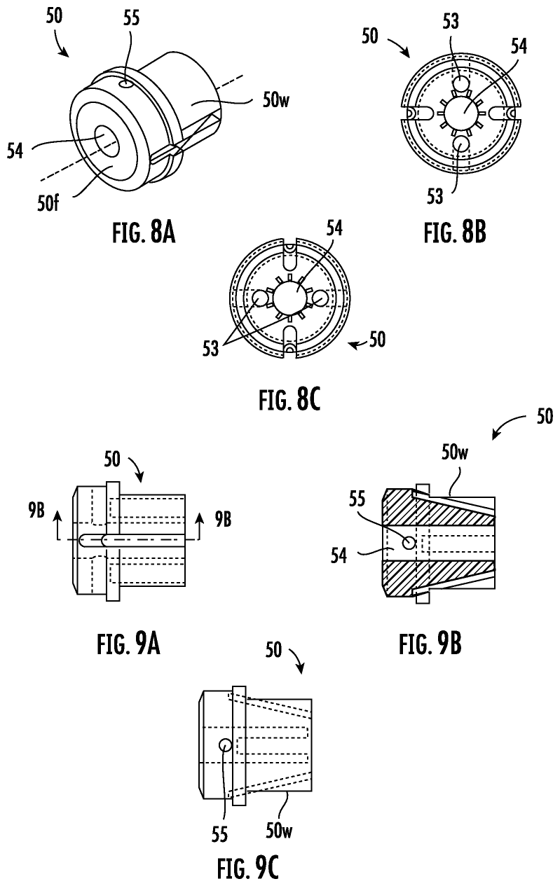

Referring to FIGS. 5, 6, 8A-8C, 9A-9C, the bearing housing 50 can have a longitudinally extending center channel 54 that holds an outer surface of the bearing/bushing 600. The bearing housing 50 can also have first and second longitudinally extending channels 53 on opposing sides of the center channel 54. The first and second channels 53 are in fluid communication with and/or hold a segment of a respective in-flow tube 133t. The bearing housing 50 can have lateral passages 55 that are in fluid communication with the ports 602 of the bearing/bushing 600, and the longitudinally extending channel 605. The lateral passages 55 can fluidly connect the in-flow path F_{I} provided by the in-flow lumens 133 with the outflow path Fo provided by the torque cable lumen 131, using the bearing/bushing 600. Seal material (such as adhesive) 155 can be used to seal outer ends of the passages 55. The lateral passages 55 can be aligned with the ports 602 of the bearing/bushing 600.

The lateral passages 55 can have an outer diameter in a range of 0.010 inches to about 0.030 inches, such as about 0.020 inches. The first and second channels 53 can have an outer diameter that is greater than the lateral passages 55, typically in a range of about 0.020 inches to about 0.030 inches, such as about 0.025 inches, in some embodiments.

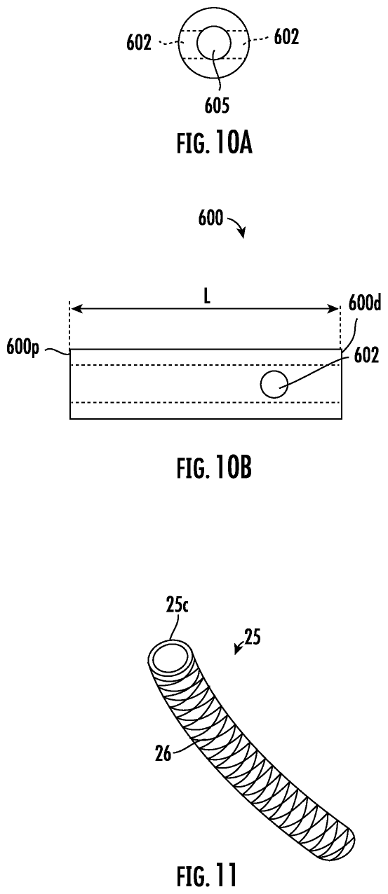

An example bearing/bushing is shown in FIGS. 10A and 10B. The bearing/bushing 600 has a center channel 605 that is sized and configured to receive a segment of the impeller shaft 140. The radially extending passages 602 reside closer to a distal end 600d of the bearing/bushing 600 than the proximal end 600p. At least one of the radially extending passages 602 can reside a distance in a range of 0.020 inches and 0.075 inches, such as about 0.050 inches, from a distal end 600d of the bearing/bushing 600, measured from a center of the radially extending passage 602.

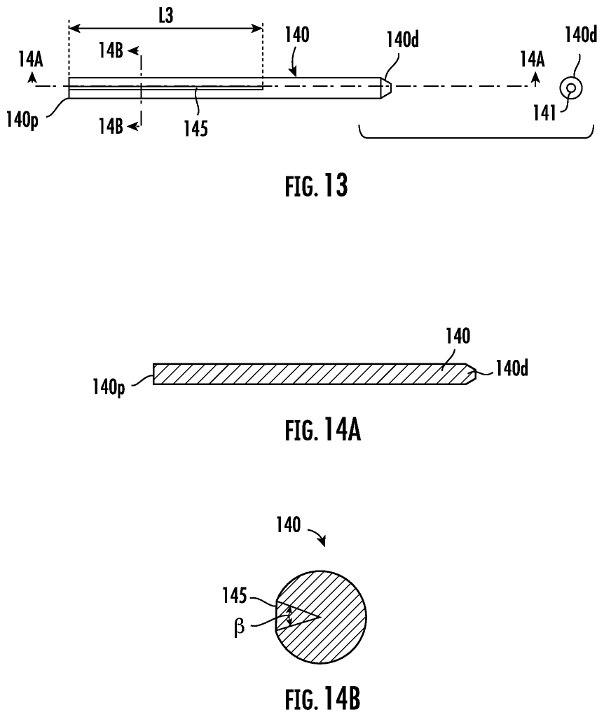

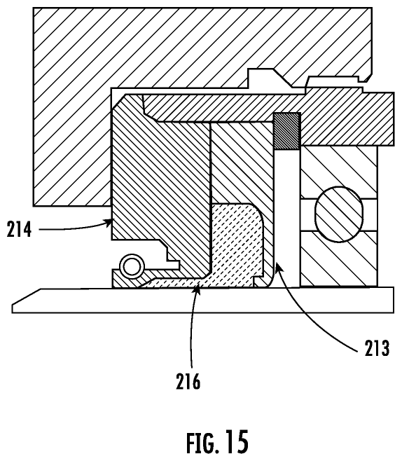

Referring to FIGS. 5, 6 and 13, the impeller shaft 140 can be configured to have a distal end 140d that is a tapered end 141. The impeller shaft 140 can have at least one outer surface segment 145 that is configured to provide a distal out-flow/liquid purge path. The at least one outer surface segment 145 can extend a sub-length of the impeller shaft 140 from a proximal end 140p to at least a medial segment. The shape/depth/width of the outer surface segment 145 can be sized to adjust for the volume of purge fluid that passes out through the shaft/bearing interface 50I. The shape/width of one or more of the at least one outer surface segment 145 can vary over a length of the impeller shaft 140. For example, a larger flow path configuration may be provided proximal to the shaft/bearing interface 50I, in a direction toward the torque cable lumen 131, relative to a size inside the body of the impeller 40.

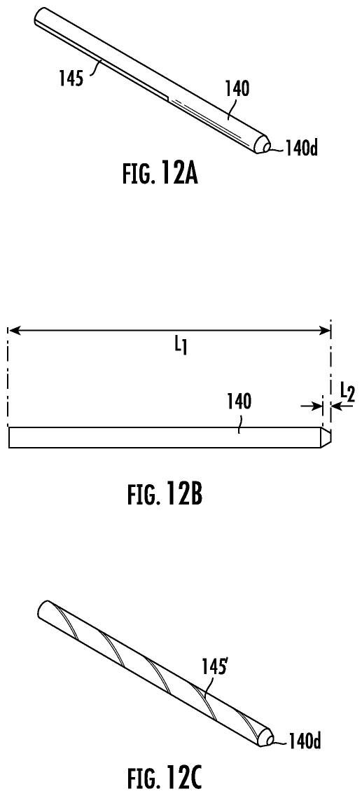

The at least one outer surface segment 145 can be flat or have a recess or other formation providing the fluid passage/path. FIG. 12C illustrates the outer surface segment 145 can be provided as a spiral groove 145′. Thus, the outer surface segment 145′ can spiral to different circumferential positions over a length.

Where the at least one outer surface segment 145 is provided as a straight recess and/or flat recess segment, such can be a single outer surface segment or a plurality of outer surface segments whereby each such recess segment or the single one segment can have a circumferential extent β that is in a range of about 45 degrees or less, typically in a range of 10-30 degrees.

The impeller shaft 140 can have an overall length L₁ that is in a range of 12-15 mm. The distal end 140d can taper inward over a small length L₂ in a range of 1 mm or less, such as 0.1 mm-0.7 mm. The at least one outer surface segment 145 can have a longitudinally extending length L₃ that is in a range of 3-8 mm, typically in a range of 7-8 mm and can start at the proximal end 140p of the impeller shaft 140. The at least one outer surface segment 145 can extend from the proximal end 140p to at least a position that is adjacent the outer face 50f of the bearing housing 50. The impeller shaft 140 can be made of hardened medical grade steel, minimum Rockwell C60. The impeller shaft 140 can be coated with a diamond like coating (DLC) for hardness and lubricity. The impeller shaft 140 can have a smooth finish that is RMS 8, in some embodiments.

Referring to FIGS. 6, 13 and 17, the at least one outer surface segment 145 can be configured to provide a discharge outflow path P_{F} as shown by the arrows for exiting proximal to the impeller 40 at the bearing housing/impeller interface 50I. The distal outflow of purge fluid can “clean” and/or provide a fluid seal at the bearing housing/impeller interface 50I so that no blood gets into the bearing/bushing 600.

Optionally, the at least one outer surface segment 145 can extend inside the body of the impeller 40 and may define a small distal out flow path P_{I} that extends toward the impeller distal end 40d as shown by the small arrows on the outside of the impeller shaft 140 and inside the impeller 40.

The blood pump 10 can be configured to provide the purge flow path without requiring seals at the distal end portion 30d of the multi-lumen shaft 30. The blood pump 10 can be configured to define an in-flow path F_{I} that merges into a cross-over path Fc, then to the out-flow path Fo using the lateral passage(s) 55 in the bearing housing 50 and radially extending passages 602 in the bearing/bushing 600, and the center channel 605 that are in fluid communication and cooperate to operate without requiring the use of seals at a distal end portion 30d of the multi-lumen shaft 30. The bearing housing 50 can cooperate with the bearing/bushing 600 to define a cross-flow segment from the in-flow lumen(s) 133 to the at least one outer surface segment 145 of the impeller shaft 140. The radially extending passages 602 can have an outer diameter in a range of 0.010 inches and 0.030 inches, such as about 0.020 inches. The center channel 605 can have a diameter that is larger than the radially extending passages 602 and the center channel 605 can be in a range of about 0.020 inches and 0.030 inches, such as about 0.026 inches.

The impeller 40 can be an expandable impeller 40. See, for example, U.S. Pat. Nos. 9,028,392, 8,079,948, pending U.S. patent application Ser. No. 17/858,615 and U.S. Provisional Patent Application Ser. No. 63/353,353, the contents of which are hereby incorporated by reference as if recited in full herein.

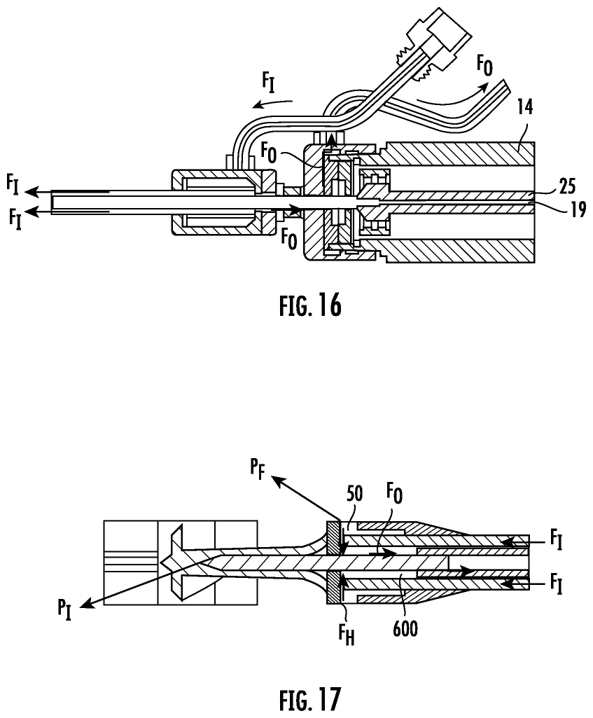

Turning now to FIGS. 16 and 17, example purge fluid directions that can be provided by the blood pump 10 are shown, in-flow “F_{I}”, out-flow “Fo”, purge fluid “P_{F}” at the impeller/bearing housing interface 50I and purge fluid “P_{I}” at the impeller shaft 140.

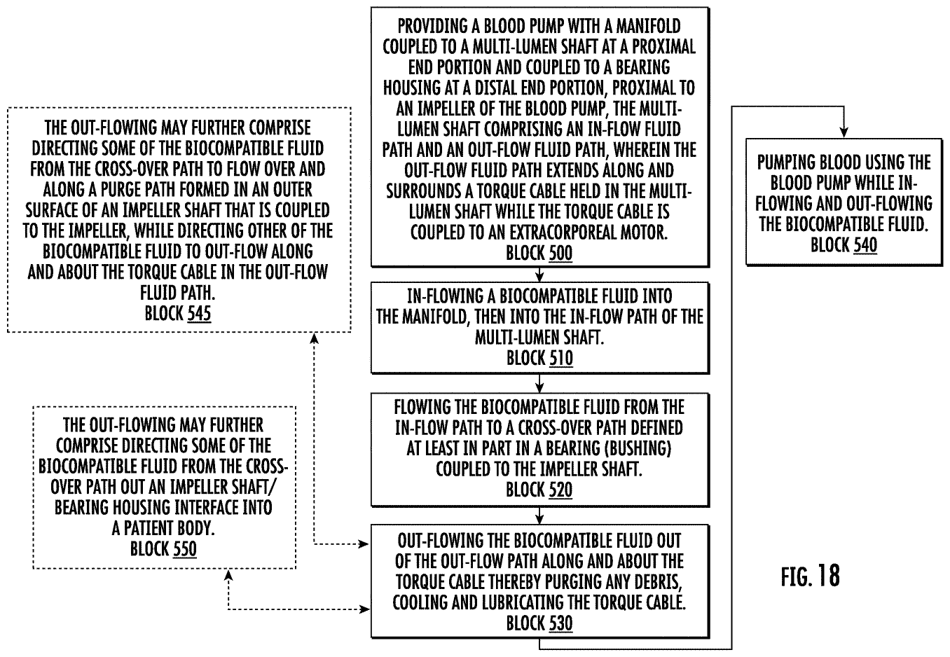

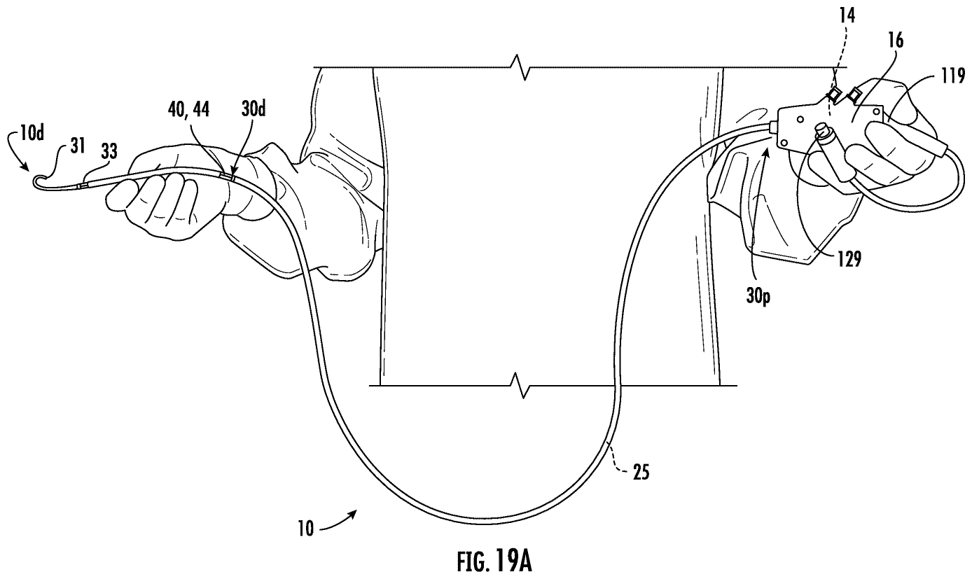

FIG. 18 illustrates actions that can be carried out for cooling/lubricating a torque cable of an intravascular blood pump is shown. A blood pump with a manifold coupled to a multi-lumen shaft at a proximal end portion and with a bearing housing at a distal end portion. The bearing housing is proximal to an impeller of the blood pump. The multi-lumen shaft comprises an in-flow fluid path and an out-flow fluid path. The out-flow fluid path extends along and surrounds a torque cable held in the multi-lumen shaft while the torque cable is coupled to an extracorporeal motor (block 500). A biocompatible fluid is in-flowed into the manifold, then into the in-flow path of the multi-lumen shaft (block 510). The biocompatible fluid from the in-flow path is then flowed to a cross-over path defined at least in part in a bearing (bushing) surrounding a segment of the impeller shaft (block 520). The biocompatible fluid is then then out-flowed out the out-flow path along and about the torque cable thereby purging any debris, cooling and lubricating the torque cable (block 530). The method also includes pumping blood using the blood pump while in-flowing and out-flowing the biocompatible fluid (block 540).

The out-flowing can include directing some of the biocompatible fluid from the cross-over path to flow over and along a purge path formed in an outer surface of an impeller shaft that is coupled to the impeller, while other volumes of the biocompatible fluid travels to the out-flowing along and about the torque cable in the out-flow fluid path (block 545).

The out-flowing may further comprise directing some of the biocompatible fluid from the cross-over path out an impeller shaft/bearing housing interface into a patient body (block 550).

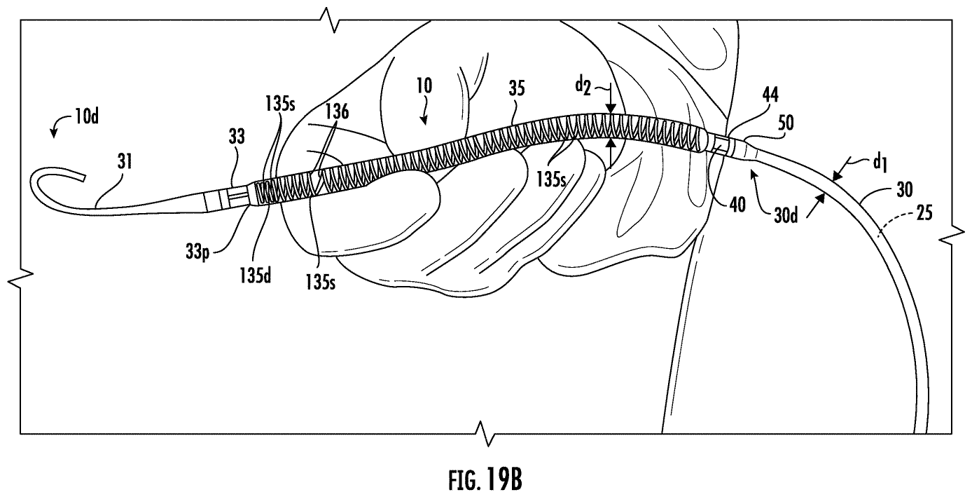

Referring to FIGS. 19A and 19B, the blood pump 10 is configured with the motor housing 16 and the motor 14 being extracorporeal. A power connector 129 can be coupled to the motor housing 16 and motor 14. The multi-lumen shaft 30 encloses a torque cable 25 and has a distal end 30d that terminates at the outlet cage 44 and/or impeller 40. The snorkel tube 35 extends between the inlet cage 33 and the impeller 40 and/or outlet cage 44 (pumped blood outlet). A snorkel 31 can be attached to the snorkel tube 35 and be positioned at a distal end 10d of the blood pump 10.

Referring to FIG. 19B, the snorkel tube 35 can comprise a coil 135 encased and/or embedded in one or more substrates of the snorkel tube 35. The snorkel tube 35 can be an extruded body with the coil 135 encased therein. The snorkel tube 35 can be a medical grade polymeric and/or co-polymeric extruded or molded body. The coil 135 can be between (inside) at least one layer of a material/substrate forming the snorkel body 35 and may be encased in a lubricious, medical grade elastomeric material. In some embodiments, the snorkel tube 35 can have an elasticity that is greater than the catheter 30 and/or can have a durometer that is less than a durometer of the catheter 30.

The coil 135 can be a single coil, multiple coils or provided, configured to provide a series of coil segments over at least a major portion of an axial length of the snorkel tube 35, shown as over an entire length thereof. The coil 135 can have a series of circumferentially extending coil segments 135s that can change in spacing/density at different locations to have increased numbers of neighboring/adjacent coil segments 135s at one or more different locations. As shown, a distal end 135d of the coil 135 can have abutting coil segments 135s and may have a smaller outer diameter relative to at least some of the more proximal coil segments 135s. The distal end 135d of the coil 135 can terminate at and be attached to a proximal end of the inlet cage 33. The coil 135 can be configured to provide at least a segment with a spiral 136 configuration. The coil 135 can be configured to have symmetrically spaced apart coil segments over some of an axial distance of the coil 135.

Still referring to FIG. 19B, the multi-lumen shaft 30 have a maximal outer diameter “d1” that is less than a maximal outer diameter “d2” of the snorkel tube 35: In some embodiments d2>1.1 d1 and d2<2d1.

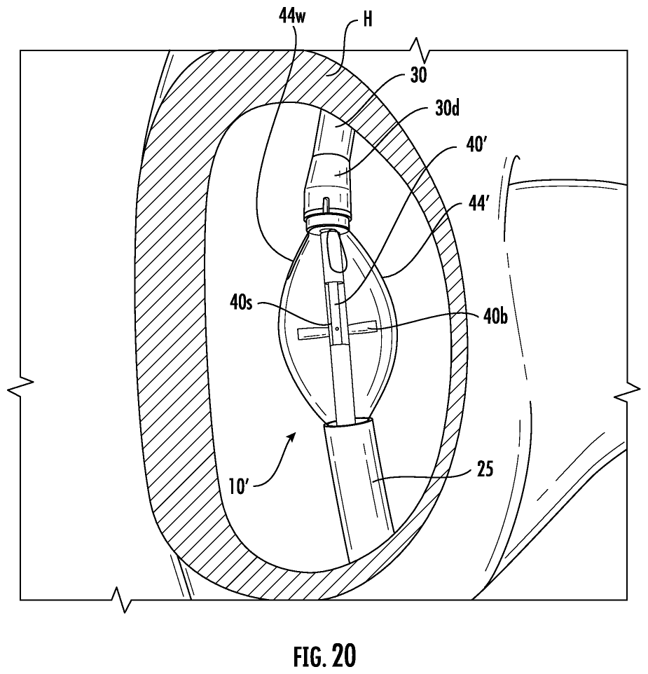

Turning now to FIG. 20, as discussed above, the impeller can be an expandable impeller 40′, shown in a heart H of a patient. The expandable impeller 40′ has a first configuration for insertion/withdrawal with the blades 44b collapsed against the shaft 40s and an operative configuration whereby the impeller blades 44b are radially projecting outward from the shaft 40s. As shown, the expandable impeller 40′ is inside an expandable (and collapsible) cage 44′ with blood outflow windows 44w. See, Further details of example expandable impeller can be found, for example, in U.S. Pat. Nos. 9,028,392, 8,079,948, pending U.S. patent application Ser. No. 17/858,615 and U.S. Provisional Patent Application Ser. No. 63/353,353, the contents of which are hereby incorporated by reference as if recited in full herein.

Embodiments of the invention provide methods of assembling and/or fabricating a blood pump. The methods include providing a multi-lumen shaft comprising a torque cable lumen and an in-flow lumen, wherein the multi-lumen shaft is flexible and elongate and configured for intra-arterial tortuous path insertion to a heart of a patient. A proximal end portion of the multi-lumen shaft is attached to a manifold. The method also includes attaching proximal end portion of a torque cable that extends a distance outside the multi-lumen shaft to a motor shaft of a motor, before or after the attachment. The method also includes attaching a proximal bushing to an impeller shaft and the torque cable and attaching a bearing/bushing to the impeller shaft. The method also includes attaching a bearing housing to the bearing/bushing and attaching an impeller to the impeller shaft.

The method can include, before inserting the proximal end portion of the torque cable to the motor shaft, inserting the proximal end portion of the torque cable through an end cap in line with the motor shaft.

The method can include attaching a bearing housing adapter to the bearing housing and outer wall of the multi-lumen shaft.

The attaching the impeller can be carried out by attaching a distal bushing to the impeller shaft and inserting the impeller shaft with the distal bushing into a channel in the impeller, then bonding the distal bushing and impeller shaft to the impeller.

The blood pump 10 can be sized and configured for trans-valvular use, such as for left and/or right ventricular assist procedures. By way of example only, such ventricular assist procedures may be employed in cardiac operations including, but not limited to, coronary bypass graft (CABG), cardiopulmonary bypass (CPB), open chest and closed chest (minimally invasive) surgery, bridge-to-transplant and/or failure-to-wean-from-bypass situations. It is to be readily understood, however, that the intravascular blood pump assembly and methods of the present invention are not to be limited to such applications. Moreover, while illustrated and described largely with reference to left-heart assist applications, it is to be readily understood that the principles of the present invention apply equally with regard to right-heart assist application, which are contemplated as within the scope of the present invention. These and other variations and additional features will be described throughout.

The blood pump 10 can be configured to pump blood through the outlet cage 44 at a rate in a range of 2-7 liters/minute over at least 6 days of continuous intravascular use while continuously providing biocompatible fluid to the in-flow path F_{I} via at least one in-flow lumen 133, then to the out-flow path Fo.

The blood pump 10 may be configured to provide axial or mixed-flow. As used herein, the term “axial flow” is deemed to include flow characteristics which include both an axial and (slight) radial component.

The blood pump 10 can be configured to provide right and/or left heart support whereby blood is deliberately re-routed through and past the right and/or left ventricle in an effort to reduce the volume of blood to be pumped by the particular ventricle. While “unloading” the ventricles in this fashion is preferred in certain instances, it is to be readily understood that the pump and cannula arrangements described herein may also be employed to “preload” the ventricles. Ventricular preloading may be accomplished by positioning the outflow cage from the pump into a given ventricle such that the pump may be employed to fill or preload the ventricle with blood. This may be particularly useful with the right ventricle. On occasion, the right ventricle is not supplied with sufficient levels of blood from the right atrium such that, upon contraction, the right ventricle delivers an insufficient quantity of blood to the pulmonary artery. This may result when the right ventricle and/or right atrium are in a stressed or distorted condition during surgery. Preloading overcomes this problem by actively supplying blood into the right ventricle, thereby facilitating the delivery of blood into the pulmonary artery. The same technique can be used to preload the left ventricle and thus facilitate the delivery of blood from the left ventricle into the aorta.

In the drawings and specification, there have been disclosed embodiments of the invention and, although specific terms are employed, they are used in a generic and descriptive sense only and not for purposes of limitation, the scope of the invention being set forth in the following claims.

Thus, the foregoing is illustrative of the present invention and is not to be construed as limiting thereof. More particularly, the workflow steps may be carried out in a different manner, in a different order and/or with other workflow steps or may omit some or replace some workflow steps with other steps. Although a few exemplary embodiments of this invention have been described, those skilled in the art will readily appreciate that many modifications are possible in the exemplary embodiments without materially departing from the novel teachings and advantages of this invention.

Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A blood pump comprising:
an extracorporeal motor housing with a motor coupled to a motor shaft;
a torque cable coupled to the motor shaft and extending longitudinally outside of the motor housing;
a multi-lumen shaft comprising a torque cable lumen sized and configured to encase at least a segment of the torque cable, wherein the torque cable lumen defines at least a portion of an out-flow fluid path that extends along and about the torque cable, wherein the multi-lumen shaft further comprises at least one fluid in-flow lumen in fluid communication with the out-flow fluid path whereby out-flow fluid in the out-flow fluid path purges debris, cools and/or lubricates the torque cable;
an impeller shaft attached to the distal end portion of the torque cable, wherein the impeller shaft comprises an outer surface configured to provide a portion of the out-flow fluid path with the outer surface configured to be in fluid communication with a distal end portion of the torque cable lumen; and
an impeller coupled to the impeller shaft.

2. The blood pump of claim 1, wherein the torque cable lumen terminates proximally a distance from a distal end of the at least one fluid in-flow lumen of the multi-lumen shaft, and wherein a proximal end portion of the impeller shaft resides adjacent to and/or inside the distal end portion of the torque cable lumen.

3. The blood pump of claim 1, wherein the motor shaft comprises an axially extending channel sized and configured to receive a portion of the torque cable.

4. The blood pump of claim 1, further comprising a motor cap coupled to the motor and a manifold coupled to a distal portion of the motor cap, wherein the motor cap comprises a fluid out-flow port in fluid communication with the torque cable lumen.

5-7. (canceled)

8. The blood pump of claim 1, further comprising a motor cap enclosing inner and outer seals that sandwich a grease pocket, wherein the motor cap is coupled to a distal end of the motor and defines a fluid out-flow port that is in fluid communication with the fluid out-flow path of the multi-lumen shaft and provides a part of the fluid out flow path that is external to the inner and outer seals and extends laterally outwardly from the motor cap.

9-12. (canceled)

13. The blood pump of claim 1, further comprising an impeller shaft that extends distally out of a bearing housing into an impeller, wherein a proximal end portion of the impeller shaft is coupled to a distal end portion of the torque cable, wherein the impeller shaft extends through a portion of a proximal bushing, and wherein the proximal end portion of the impeller shaft and the proximal bushing are attached to the torque cable to thereby couple the torque cable to the impeller shaft.

14. The blood pump of claim 1, further comprising a bearing housing that holds a bearing that surrounds a portion of the impeller shaft, and wherein the bearing comprises at least one radially extending open channel that is in fluid communication with a fluid in-flow path provided by the at least one in-flow lumen of the multi-lumen shaft to define a cross-over portion for purge fluid to cross-over from the at least one in-flow lumen to the out-flow fluid path and with a portion of the purge fluid from the cross-over portion flows between the impeller shaft and the bearing whereby the bearing acts as a hydrostatic bearing thereby inhibiting contact between the bearing and the impeller shaft.

15. The blood pump of claim 14, further comprising axially spaced apart proximal and distal bushings residing on opposing end portions of the bearing, wherein the proximal bushing is attached to the impeller shaft and the torque cable, and wherein the distal bushing is attached to the impeller shaft distal to the bearing housing.

16. The blood pump of claim 15, wherein the proximal and distal bushings are configured to define thrust bushings, wherein the impeller shaft extends through the proximal and distal bushings and the bearing, and wherein the proximal bushing comprises a distal flange and the distal bushing comprises a proximal flange and the bearing is fixed in position between the distal flange and the proximal flange.

17. (canceled)

18. The blood pump of claim 1, wherein the torque cable comprises an axially extending lumen, and wherein the axially extending lumen of the torque cable holds at least a segment of at least one support wire.

19. (canceled)

20. The blood pump of claim 14, wherein the bearing housing holds tube segments forming the at least one in-flow lumen of the multi-lumen shaft and comprises lateral passages that fluidly connect fluid from the tube segments across the bearing housing to the at least one radially extending open channel in the bearing, and wherein outer end portions of the at least one radially extending open channel are sealed.

21. A method of cooling/lubricating a torque cable of an intravascular blood pump, comprising:
providing a blood pump with a manifold coupled to a multi-lumen shaft at a proximal end portion and with a bearing housing adjacent a distal end portion of the multi-lumen shaft, proximal to an impeller of the blood pump, the multi-lumen shaft comprising an in-flow fluid path and an out-flow fluid path, wherein the out-flow fluid path extends along and surrounds a torque cable held in the multi-lumen shaft while the torque cable is coupled to an extracorporeal motor;
in-flowing a biocompatible fluid into the manifold, then into the in-flow path of the multi-lumen shaft; then
flowing the biocompatible fluid from the in-flow path to a cross-over path defined at least in part in a bearing surrounding part of an impeller shaft coupled to an impeller;
then out-flowing the biocompatible fluid out of the out-flow path along and about the torque cable thereby purging any debris, cooling and lubricating the torque cable; and
pumping blood using the blood pump while in-flowing and out-flowing the biocompatible fluid.

22. The method of claim 21, wherein the flowing further comprises directing some of the biocompatible fluid from the cross-over path to flow over and along a purge path formed in the impeller shaft that is coupled to the impeller, while directing other of the biocompatible fluid to the out-flowing along and about the torque cable in the out-flow fluid path.

23. The method of claim 21, wherein the flowing further comprises directing some of the biocompatible fluid from the cross-over path out an interface between the impeller shaft and the bearing housing interface into a patient body while directing other of the biocompatible fluid to the out-flowing along and about the torque cable in the out-flow fluid path.

24-30. (canceled)

31. The blood pump of claim 1, wherein the impeller shaft comprises
a shaft body that extends in an axial direction, the shaft body comprising a proximal end portion and a spaced apart opposing distal end portion, wherein the shaft body comprises an outer surface with at least one flat segment or recess that extends over at least a portion of a length of the shaft body to thereby define a purge fluid passage.

32. The blood pump of claim 31, wherein the at least one flat segment or recess defines a flat segment on the outer surface.

33. The blood pump of claim 31, wherein the at least one flat segment or recess is a single recess.

34. The blood pump of claim 31, wherein the at least one flat segment or recess is a spiral groove.

35. The blood pump of claim 32, wherein the flat segment extends circumferentially a distance of 0.010 mm to 0.025 mm about the outer surface.

36. (canceled)

37. The blood pump of claim 31, wherein the impeller shaft has a length in a range of 10-15 mm, and wherein the at least one flat segment or recess extends a distance that is at least 50% of the length of the shaft body from a proximal end thereof to a location proximal to a distal end of the shaft body.

38-41. (canceled)

42. The blood pump of claim 1, further comprising:
a distal bushing arranged to surround and attach to an outer surface of a portion of the impeller shaft and is received in a channel of the impeller and attached to a wall surface of the channel of the impeller;
a proximal bushing arranged to surround and attach to an outer surface of a portion of the impeller shaft at a location proximal to the distal bushing; and
a bearing surrounding a segment of the impeller shaft and residing between the distal and proximal bushings,
wherein a distal end segment of the torque cable is held inside the proximal bushing, attached to the proximal bushing at a spaced apart location from the impeller shaft.

43. The blood pump of claim 42, further comprising a bearing housing surrounding the bearing, wherein the bearing housing is fixed to an outer wall of a catheter body providing the multi-lumen shaft.

44-45. (canceled)

46. The blood pump of claim 42, wherein the proximal and distal bushings are configured to define thrust bushings, wherein the bearing is held inside a bearing housing that is fixed to an outer wall of a catheter providing the multi-lumen shaft and the proximal and distal bushings are configured to keep the impeller axially stationary preventing axial movement relative to the bearing.

47. The blood pump of claim 43, wherein the bearing housing has an outer surface facing the impeller and defines an interface thereat, and wherein the interface is configured to eject purge fluid toward the impeller.

48. The blood pump of claim 42, wherein the torque cable comprises a center channel that holds at least one support wire, and wherein the proximal bushing is crimped onto a distal end portion of the torque cable and one of the at least one support wire at a first location and is crimped onto the impeller shaft at a second location that is distal to the first location.

49-53. (canceled)

54. The blood pump of claim 43, wherein the bearing housing has an external surface that faces the impeller and defines a bearing and impeller interface, wherein a first amount of purge fluid is configured to exit at the bearing and impeller interface while maintaining a sufficient fluid pressure that prevents blood from entering the bearing housing while a larger second amount of purge fluid is directed back to an out-flow path provided by the out-flow lumen of a multi-lumen shaft.

55-57. (canceled)

58. The blood pump of claim 1, wherein the blood pump further comprises
inner and outer seals that are coupled together and sandwich a grease pocket, wherein the motor shaft with the torque cable therein extends through the inner and outer seals, and wherein the torque cable is sized and configured to extend a distance outside the motor shaft and outside of the motor housing.

59. The blood pump of claim 58, wherein the inner seal is configured a memory lip seal and the outer seal is configured as a cooperating a stainless-steel spring that applies pressure to the torque shaft, and wherein the grease pocket comprises food grade grease.

60. The blood pump of claim 58, wherein the motor comprises a motor end cap that defines a fluid out-flow port that is in fluid communication with the fluid out-flow path of a multi-lumen shaft that encloses the torque cable and that provides at least in part, the fluid out-flow path that is external to the inner and outer seals and that extends laterally outwardly from the motor cap to the fluid out-flow port.

61. (canceled)

62. The blood pump of claim 1, wherein the motor shaft comprises a cylindrical metal body with a center channel that is open and sized and configured to receive a flexible torque cable, wherein the cylindrical body has a maximal outer diameter that is in a range of 0.050 inches to 0.100 inches, and has an outer surface comprising a diamond like coating (DLC) to provide hardness and lubricity.

63. A blood pump comprising:
an extracorporeal motor housing with a motor coupled to a motor shaft, wherein the motor shaft comprises an axially extending channel;
a torque cable coupled to the motor shaft and extending longitudinally a distance outside the blood pump housing;
a multi-lumen shaft comprising a torque cable lumen sized and configured to encase a segment of the torque cable and define at least a portion of a purge fluid out-flow path, wherein the multi-lumen shaft further comprises at least one in-flow lumen;
an impeller coupled to a distal end of the torque cable; and
at least one support wire that extends through at least a segment of the axially extending channel of the torque cable.

64. The blood pump of claim 63, wherein the at least one support wire extends through the motor shaft and extends proximally to terminate in and/or to extend out of a proximal end of the torque cable.

65. The blood pump of claim 63, wherein the torque cable extends through an entire length of the motor shaft.

66. (canceled)

67. The blood pump of claim 63, wherein the at least one support wire terminates a distal to a manifold coupled to the motor, and wherein the manifold defines a portion of a fluid in-flow path that is in fluid communication with the at least one in-flow lumen.

68. The blood pump of claim 63, wherein the at least one support wire is provided as a first support wire and a second support wire, the second support wire configured to extend through the torque cable, distally of the first support wire.

69-70. (canceled)

71. The blood pump of claim 63, further comprising a motor cap coupled to the motor and a manifold coupled to the motor cap, wherein the motor cap comprises a fluid out-flow port in fluid communication with the out-flow path.

72. The blood pump of claim 71, further comprising a manifold coupled to the motor cap and comprising a fluid in-flow port in fluid communication with the at least one in-flow lumen, wherein the manifold surrounds a sub-length of the multi-lumen shaft.

73. (canceled)

74. The blood pump of claim 72, wherein the multi-lumen shaft comprises a proximal end portion that resides in the manifold, wherein the proximal end portion of the multi-lumen shaft comprises an outer wall with fluid intake ports in fluid communication with the at least one in-flow lumen, wherein the blood pump further comprises a fluid in-flow conduit attached to the manifold at one end and to a fluid in-flow connector at an opposing end.

75. The blood pump of claim 63, further comprising a motor cap enclosing inner and outer seals that sandwich a grease pocket, wherein the motor cap defines an end cap of the motor housing and provides a fluid out-flow port that is in fluid communication with a fluid out-flow path of the multi-lumen shaft, wherein the motor cap defines a part of an out-flow path of the blood pump that is external to the inner and outer seals and that extends laterally outwardly from the motor cap.

76-80. (canceled)

81. The blood pump of claim 63, further comprising an impeller shaft that is coupled to the impeller and that extends distal out of a bearing housing.

82. The blood pump of claim 63, further comprising an impeller shaft that is attached to the impeller and that has at least one exterior surface segment that defines a portion of the out-flow fluid path.

83. The blood pump of claim 63, further comprising a bearing housing that holds a bearing that surrounds a segment of the impeller shaft, wherein the bearing comprises at least one radially extending channel that is in fluid communication with a fluid in-flow path provided at least in part by the at least one in-flow lumen of the multi-lumen shaft to define at least one cross-over fluid path, and wherein the bearing acts as a hydrostatic bearing.

84. The blood pump of claim 83, further comprising axially spaced apart proximal and distal bushings residing on opposing sides of the bearing, wherein the proximal bushing is attached to the impeller shaft, the torque cable and the at least one support wire, and wherein the distal bushing is attached to the impeller shaft outside the bearing housing.

85. The blood pump of claim 84, wherein the proximal and distal bushings are configured to define thrush-washers with respective radially extending flanges with the bearing therebetween.

86. (canceled)

87. The blood pump of claim 63, further comprising an impeller shaft coupled to the impeller and to the torque cable, wherein the impeller shaft defines a portion of the fluid out-flow path in fluid communication with a cross-over fluid path provided by a bearing that surrounds a portion of the impeller shaft.

88. The blood pump of claim 87, wherein the bearing surrounds a portion of an impeller shaft that is coupled to the impeller, wherein biocompatible purge fluid from the at least one in-flow lumen travels distally through holes in the bearing providing the cross-over fluid path then along a portion of the impeller shaft into and along the torque cable lumen thereby purging debris and/or cooling and lubricating the torque cable.

89. The blood pump of claim 88, wherein the bearing housing has an external surface that faces the impeller and defines a bearing and impeller interface, wherein a first amount of purge fluid is configured to exit at the bearing and impeller interface into a patient while maintaining a sufficient fluid pressure that prevents blood from entering the bearing housing while a greater amount of purge fluid is configured to exit the blood pump along an outflow path provided by the multi-lumen shaft.

90. The blood pump of claim 63, wherein the blood pump is configured to pump blood through the outlet cage at a rate in a range of 2-7 liters while continuously providing biocompatible fluid to the at least one in-flow lumen, then to the out-flow path.

91-101. (canceled)
